(19) United States
(12) Patent Application Publication
(10) Pub. No.: US 2026/0148846 A1
(43) Pub. Date: May 28, 2026

(54) SYSTEMS AND METHODS OF OBTAINING VITALS VIA PHONE CALL

(71) Applicant: New York University, New York, NY (US)

(72) Inventors: Nyamitse-Calvin Mahinda, New York, NY (US); Harsh Sonthalia, New York, NY (US); Tae Hong Park, New York, NY (US)

(21) Appl. No.: 19/124,110

(22) PCT Filed: Oct. 25, 2023
(86) PCT No.: PCT/US2023/077746

§ 371 Date: Apr. 24, 2025

Related U.S. Application Data
(60) Provisional application No. 63/380,817, filed on Oct. 25, 2022.

(51) Int. Cl. G16H 40/63

(52) U.S. Cl. CPC G16H 40/63

(57) ABSTRACT

A system for calculating vitals via phone call comprises a computing system communicatively connected to a telephonic communication system, comprising a processor and a non-transitory computer-readable medium with instructions stored thereon, which when executed by the processor, host an application programming interface (API) configured to intercede into or interface with a call on the telephonic communication system to perform steps via the computing system comprising requesting a patient utter a sound for a set duration, capturing an audio file or audio signal, and/or calculating vitals based on the audio file or audio signal. Related methods and non-transitory computer readable medium are also disclosed.

### CROSS-REFERENCE TO RELATED APPLICATIONS

This application claims priority to U.S. provisional application No. 63/380,817 filed on Oct. 25, 2022, incorporated herein by reference in its entirety.

### BACKGROUND OF THE INVENTION

The healthcare industry evolution has recently been catalyzed with innovative technologies. This has created a secondary avenue for healthcare delivery namely Telehealth/Telemedicine. However, there are some significant limitations that directly impact patient care. Current technologies being utilized in Telehealth require end-users to be knowledgeable in the technology. This presents a challenge to certain demographics attempting to utilize this avenue such as elderly populations, and individuals facing socioeconomic disparities. In addition, the majority of providers are forced to make intervention decisions on their own gestalt due to limited accurate information i.e., vital signs. A key challenge is to help healthcare providers access key vitals quickly, easily, and accurately when they need them in order to prevent unnecessary patient readmission to the hospital/clinic. Furthermore, there is a lack of real-time, accurate data for triage processes and route intervention.

Thus, there is a need in the art for improved systems and methods for obtaining patient vitals remotely.

### SUMMARY OF THE INVENTION

Some embodiments of the invention disclosed herein are set forth below, and any combination of these embodiments (or portions thereof) may be made to define another embodiment.

In one aspect, a system for calculating vitals via common “phone calls” comprises a computing system communicatively connected to a telephonic communication system, comprising a processor and a non-transitory computer-readable medium with instructions stored thereon, which when executed by the processor, host an application programming interface (API) configured to intercede into or interface with a call on the telephonic communication system to perform steps via the computing system comprising requesting a patient utter a sound for a set duration, capturing an audio file or an audio signal, and/or calculating vitals based on the audio file or audio signal.

In one embodiment, the step of calculating vitals based on the audio file or audio signal comprises trimming the audio file or audio signal to a set timeframe or duration, performing digital signal processing including time-domain, frequency-domain, and/or spectral analysis such as a short time Fourier transform to obtain a spectrogram, analyzing the waveform and the spectrogram for patterns in a defined frequency and/or magnitude range, graphing an electrocardiogram (ECG) based on the analysis, passing the ECG through a filtering process such as a low pass filter to produce a filtered ECG, detecting peaks or salient resonance points in the filtered ECG signal to obtain frequency values, and/or calculating a heart rate based on the frequency values obtained.

In one embodiment, the step of calculating vitals based on the audio file or audio signal further comprises requesting utterance of vowel at specific frequency range and/or energy level with or without an example template, and/or computing robustness of the vowel utterance by comparing it to the example template.

In one embodiment, the calculated vitals comprise at least one of heart rate, lung capacity, oxygen saturation, ECG trace, slurred speech, blood pressure, and mean arterial pressure.

In one embodiment, the API further performs steps via the computing system comprising providing the calculated vitals to a medical practitioner, and/or removing itself from the call.

In one embodiment, the API further performs steps via the computing system comprising initiating an automated telephone call, providing a clinical questionnaire via the automated telephone call or a text message, obtaining responses to the clinical questionnaire via the automated telephone call or the text message, and/or providing the calculated vitals and the responses to the clinical questionnaire to a medical practitioner.

In one embodiment, the system further comprises a database communicatively connected to the computing system.

In one embodiment, the API via the computing system is further configured to store the audio file or audio signal, feature vectors, algorithmic parameters, and/or calculated vitals on the database.

In another aspect, a method for obtaining vitals via phone call comprises providing a test tone or an appropriate synthetic human vocal sound such as, but not limited to, a vowel sound through the user's phone to assist the user in articulating a quasi-normalized vowel sound in both “pitch” (fundamental frequency) and “loudness” (amplitude) as a form of signal conditioning prior signal analysis. This embodiment includes a fundamental frequency detector and amplitude envelope detector to determine if the vocal utterances have been properly articulated including user feedback to “try again,” “louder,” “softer,” etc. The signal is then subject to low frequency analysis via time-domain and frequency domain analysis, filtering, and low frequency oscillation detection for automatic, remote heartbeat pulse detection.

In another aspect, a method for obtaining vitals via phone call comprises using the on-board microphone of the user's device, such as a smartphone and placing in near the heart whereby exploiting superior acoustic sound propagation solids and fluids when compared to propagation the air. In this embodiment, external environmental noise is blocked while internal heartbeat/pulse sounds maximally captured by the mic. The signal is then subject to low frequency analysis via time-domain and frequency domain analysis, filtering, and low frequency oscillation detection for automatic, remote heartbeat pulse detection.

In another aspect, a method for obtaining vitals via phone call comprises providing the system as described above, and interceding into or interfacing with a phone call via an application programming interface (API) of the computing system to perform steps via the computing system comprising, sending a request to a patient to utter a sound for a set duration, capturing an audio file or audio signal, and/or calculating vitals based on the audio file or audio signal.

In one embodiment, the step of calculating vitals based on the audio file or audio signal comprises trimming the audio file or audio signal to a set timeframe, performing digital signal processing including time-domain, frequency-domain, and/or spectral analysis such as a short time Fourier transform to obtain a spectrogram, analyzing the waveform and the spectrogram for patterns in a defined frequency and/or magnitude range, graphing an electrocardiogram (ECG) based on the analysis, passing the ECG through a filtering process such as a low pass filter to produce a filtered ECG, detecting peaks or salient resonance points in the filtered ECG signal to obtain frequency values, and/or calculating a heart rate based on the frequency values obtained.

In one embodiment, the step of calculating vitals based on the audio file or audio signal further comprises requesting utterance of vowel at specific frequency range and/or energy level with or without an example template, and computing robustness of the vowel utterance by comparing it to the example template.

In one embodiment, the calculated vitals comprises at least one of heart rate, lung capacity, oxygen saturation, ECG trace, slurred speech, blood pressure, and mean arterial pressure.

In one embodiments, the API further performs steps via the computing system comprising providing the calculated vitals to a medical practitioner, and/or removing itself from the call.

In one embodiment, the API further performs steps via the computing system comprising initiating an automated telephone call, providing a clinical questionnaire via the automated telephone call or a text message, obtaining responses to the clinical questionnaire via the automated telephone call or the text message, and/or providing the calculated vitals and the responses to the clinical questionnaire to a medical practitioner.

In one embodiment, the API via the computing system is further configured to identify slurring, patterns or abnormalities in the audio file or audio signal.

In one embodiment, the API via the computing system is further configured to calculate a score indicative of trauma, infection, or cardiac distress.

In one embodiment, the API via the computing system is further configured to provide the score to a medical practitioner.

In one embodiment, the API via the computing system automatically intercedes the call.

In one embodiment, the API via the computing system intercedes the call after an operator initiates the API to intercede.

In one embodiment, the API via the computing system is further configured to initiate clinical follow-up notes.

In another aspect, a non-transient computer readable medium storing instructions that, when executed by a computing system, cause the computer system connected to a telephonic communication system to host an application programming interface (API) configured to intercede into or interface with a call on the telephonic communication system to perform steps via the computing system comprising, requesting a patient utter a sound for a set duration, capturing an audio file or audio signal, and/or calculating vitals based on the audio file or audio signal.

In one embodiment, the calculated vitals comprises at least one of heart rate, lung capacity, oxygen saturation, ECG trace, slurred speech, blood pressure, and mean arterial pressure.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts an exemplary computing environment in which aspects of the invention may be practiced in accordance with some embodiments.

FIG. 2 is a block diagram depicting an exemplary system for obtaining vitals via phone call in accordance with some embodiments.

FIG. 3 is a flow chart depicting a method for obtaining vitals via phone call in accordance with some embodiments.

FIG. 4 is a flow chart depicting a method for remote patient monitoring in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of obtaining vitals via phone call. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are systems and methods of obtaining vitals via phone call.

The disclosed system and methods focus on capturing patient vital signs through audio modalities to prevent unnecessary readmission to the hospital/clinic in the long-term as well as improve the triage process. The approach further innovates the current RPM (remote patient monitoring) paradigm and is engineered to improve the quality of the virtual triage process. This meets the need for patients either at home with or without virtual management, or those who need hospitalization.

The solution requires the patient to simply vocalize a lengthened single syllable on a phone call, as prompted, which is then analyzed. Using a similar principle to the method of Eulerian Video Magnification, which involves spatial decomposition and temporal filtering on an audio input, the patient's heart rate and other vitals are extracted from the audio file or audio signal and submitted to the healthcare provider. Based on the heart rate and vitals extracted, it is further possible to obtain a range of other key vitals such as lung capacity and also to determine SpO₂ (oxygen saturation).

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C #, Objective-C, Java, Javascript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

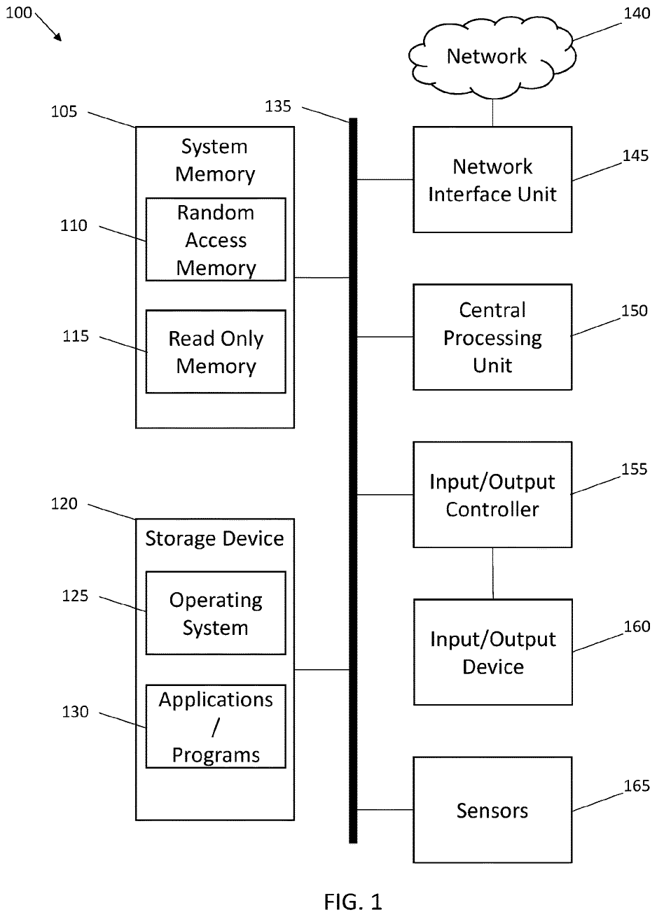

FIG. 1 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1 depicts an illustrative computer architecture for a computer 100 for practicing the various embodiments of the invention. The computer architecture shown in FIG. 1 illustrates a conventional personal computer, including a central processing unit 150 (“CPU”), a system memory 105, including a random-access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data.

The storage device 120 is connected to the CPU 150 through a storage controller (not shown) connected to the bus 135. The storage device 120 and its associated computer-readable media, provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including a keyboard, a mouse, a touchscreen, a camera, a microphone, a controller, a joystick, or other type of input device. Similarly, the input/output controller 155 may provide output to a display screen, a printer, a speaker, or other type of output device. The computer 100 can connect to the input/output device 160 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files or signals may be stored in the storage device 120 and RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. These sensors 165 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### System for Obtaining Vitals via Phone Call

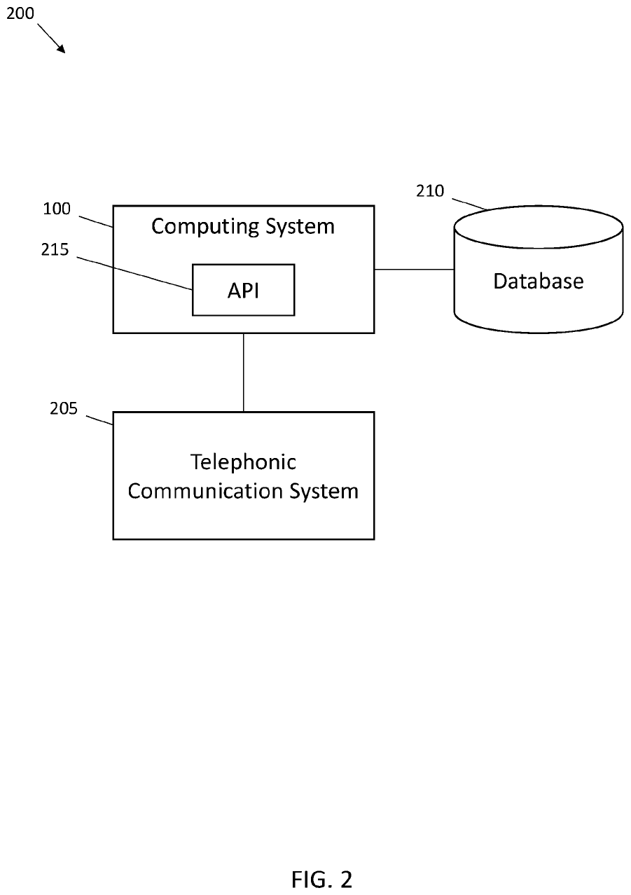

Referring now to FIG. 2, an exemplary system for obtaining vitals via phone call 200 is shown. In some embodiments, the system 200 is configured to perform remote patient monitoring (RPM) and/or emergency triage. In some embodiments, the system 200 includes a computing system 100 communicatively connected to a telephonic communication system 205. The telephonic communication system 205 can be any suitable telephonic system, including wireless and/or wired, and can utilize standard telephonic protocols. In some embodiments, the computing system 100 includes a processor and a non-transitory computer-readable medium with instructions stored thereon, which when executed by the processor, host an application programming interface (API) 215 configured to intercede into or interface with a call on the telephonic system 205.

In some embodiments, the interceding is performed at the switch/exchange level using an existing public switched telephone network (PSTN) infrastructure for handoffs such as, for example, call waiting and sequential calls. In some embodiments, the interceding is performed at the private branch exchange (PBX) level local to an entity such as a hospital or medical facility. In some embodiments, the interceding is performed via a voice over internet protocol (VoIP). In some embodiments, the interceding is performed via an application on a mobile telephone, smart phone, or any other suitable smart portable device. In some embodiments, the interceding is performed via an application on a desk phone, computer, or similar device. In some embodiments, the interceding is performed at a cloud based switch/exchange level such as Twilio, for example.

In some embodiments a database 210 configured to store audio files, audio signals, and/or vitals results is communicatively connected to the computing system 100. In some embodiments, the database 210 provides for advantages in patient privacy and ease of use, as vitals data is only stored on the database and not on a patient's personal phone. In some embodiments, database 210 may comprise or may form a part of an electronic medical record (EMR) database.

In some embodiments, the API 215 and computing system 100 are configured to perform steps for obtaining vitals via phone call including requesting a patient utter a sound for a set duration, capturing an audio file or audio signal, and/or calculating vitals based on the audio file or audio signal. In some embodiments, the system 200 prompts a patient to utter a sound for a duration in the range of 1 second to 20 seconds, 5 seconds to 10 seconds, 6 seconds to 8 seconds, about 7 seconds, or any other suitable duration. In some embodiments, the system 200 prompts a patient to utter a vowel sound.

In some embodiments the calculated vitals include heart rate, lung capacity, oxygen saturation, ECG trace, slurred speech, blood pressure, mean arterial pressure, or other suitable vitals.

In some embodiments, the API 215 and computing system 100 are further configured to perform steps including providing the calculated vitals to a medical practitioner, and/or removing itself from the call.

In some embodiments, the API 215 and computing system 100 are further configured to perform steps including initiating an automated telephone call, providing a clinical questionnaire via the automated telephone call or a text message, obtaining responses to the clinical questionnaire via the automated telephone call or the text message, and/or providing the calculated vitals and the responses to the clinical questionnaire to a medical practitioner.

The system 200 is advantageous in that heart rate speed and variability statistics can be calculated from traditional phone calls without the need for patients to possess or install any software or hardware. Heart rate data can be captured during existing phone calls with providers, for instance when a patient calls and requests urgent or emergency services and must be triaged among primary care, urgent care, and emergency department services. Heart rate data can also be captured asynchronously for provider review as part of the post-discharge protocol.

For instance, several automated check-ins with a patient post-discharge provides for results which are then reviewed by a provider during their scheduled follow-up. In some embodiments, the vitals results are visible to the patient and/or the doctor on respective dashboards. Depending on the critical nature of the health of each individual patient, the doctor can then decide on the appropriate action that needs to be taken for that particular patient.

In some embodiments, the system 200 is configured for remote patient monitoring. The system 200 can monitor patients pre-hospitalization and/or post-hospitalization and provide ongoing objective data to clinicians working in telehealth settings.

In some embodiments, the system 200 can be configured as a clinical follow-up tool, where the system is configured to keep track of trends and help clinicians revise patients'treatment plans after follow-ups or check-ins.

In some embodiments, the system 200 is configured for implementation in urgent or emergency service requests. For example, when a patient makes an inbound phone call, the system 200 can calculate and provide heart rate and other vitals to the provider in real-time.

In some embodiments, the system 200 can be configured to allow providers and medical practices to send outbound calls to patients to enroll and on-board patients onto the vital audio system. In some embodiments, providers can request the system 200 to call patients at a specified cadence and times, and measure heart rate and other vitals until the time a provider reviews the patient's vital data log. In some embodiments, the system may be configured to send out alerts when vitals are out of a specified range (i.e., high and low measurements). In some embodiments, providers can make outbound calls for patient check-ins and follow-ups as needed based on objective data.

In some embodiments, the API 215 comprises a plugin, such as a Twilio or EMR plugin, that resides as an application layer in a providers' existing inbound and follow-up telephone workflows. In some embodiments, the API 215 injects itself into the telephone workflow.

### Calculating Vitals Based on Audio File or Audio Signal

Presented herein is an exemplary process for calculating vitals based on an audio file or audio signal. An audio file (.wav, .mp3, or similar) or audio signal including vowel speech of a set duration is truncated to a desired timeframe duration, for example, to 6 to 8 seconds or other suitable duration. With the truncated audio file or audio signal, P and T waves are conditioned using signal processing, modulation, and/or filtering processing such as lowpass filtering with a desired cutoff frequency, for example, around 40 Hz. A time-domain, frequency-domain, and/or spectral analysis procedure such as a Short Time Fourier Transform (STFT) is used to create a frequency-domain representation to convert the vowel speech of the audio file or audio signal to an Electrocardiogram (ECG). The spectrogram is then searched for frequency values in a defined range, for example, 200 Hz to 6 KHz. The data is logged in memory and/or saved in a file (.csv, or similar) and is then used to graph the Electrocardiogram (ECG). The ECG is then passed through additional filtering processes such as a low pass filter which results in a filtered ECG chart that is similar to the ones that are displayed on ECG monitors. The filtered ECG chart then undergoes resonance peak analysis to render a heart rate based on changes in frequency patterns.

For further information and details on an exemplary calculation of vitals see Mesleh et al., “Heart rate extraction from vowel speech signals”. JOURNAL OF COMPUTER SCIENCE AND TECHNOLOGY 27(6): 1243-1251 Nov. 2012. DOI 10.1007/s11390-012-1300-6, incorporated herein by reference in its entirety.

In some embodiments, the API 215 utilizes a plug-in communication tool (for example, Twilio) for integration into electronic medical records (EMRs). This allows the system 200 to integrate the captured audio data and results into the EMRs.

In some embodiments, machine learning and/or artificial intelligence is utilized to more robustly capture and make measurements and calculations of the vitals. In some embodiments, machine learning and/or artificial intelligence is utilized to eliminate or reduce environmental noise and disturbances in the captured audio data to improve the measurements and calculations of the vitals.

In some embodiments, calculating vitals based on the audio file or audio signal further includes requesting utterance of a vowel sound at a specific frequency range and/or energy level with or without an example template, and/or computing robustness of the vowel utterance by comparing it to the example template.

### Method for Obtaining Vitals via Phone Call

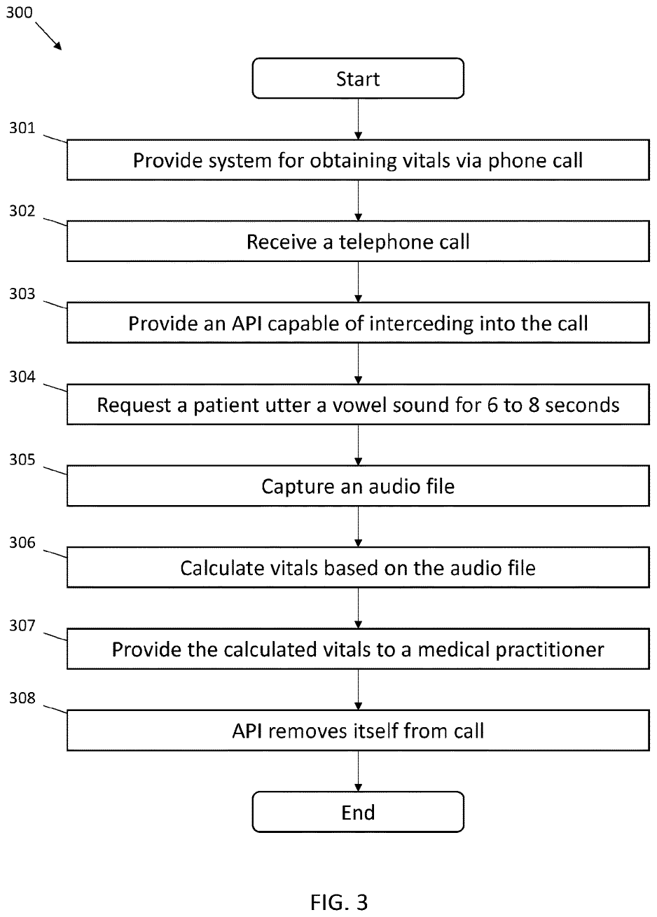

Referring now to FIG. 3, an exemplary method 300 for obtaining vitals via phone call is shown. The method 300 starts at Operation 301 where a system for obtaining vitals via phone call such as system 200 is provided. At Operation 302 a telephone call is received. At Operation 303 an API 215 configured to intercede into the call is provided.

In some embodiments, the interceding is performed at the switch/exchange level using an existing public switched telephone network (PSTN) infrastructure for handoffs such as, for example, call waiting and sequential calls. In some embodiments, the interceding is performed at the private branch exchange (PBX) level local to an entity such as a hospital or medical facility. In some embodiments, the interceding is performed via a voice over internet protocol (VoIP). In some embodiments, the interceding is performed via an application on a mobile telephone, smart phone, or any other suitable smart portable device. In some embodiments, the interceding is performed via an application on a desk phone, computer, or similar device. In some embodiments, the interceding is performed at a cloud based switch/exchange level such as Twilio, for example.

At Operation 304 a request is sent to a patient to utter a vowel sound for a set duration such as, for example, a duration in the range of 1 second to 20 seconds, 5 seconds to 10 seconds, 6 seconds to 8 seconds, about 7 seconds, or any other suitable duration. At Operation 305, an audio file or audio signal is captured. The audio file can be any suitable audio file (.wav, .mp3, or similar) or any suitable audio signal and includes vowel speech of a set duration. Suitable vowel sounds include a short ‘a’ sound, a long ‘a’ sound, a short ‘e’ sound, a long ‘e’ sound, a short ‘i’ sound, a long ‘i’ sound, a short ‘o’ sound, a long ‘o’ sound, a short ‘u’ sound, a long ‘u’ sound, or any combination of these. In some embodiments, multiple requests to utter multiple different vowel sounds may be sent to the patient serially in order to collect multiple readings for analysis.

At Operation 306 vitals are calculated based on the audio file or audio signal. In some embodiments, vitals are calculated as described above. In some embodiments the calculated vitals include one or more of heart rate, lung capacity, oxygen saturation, ECG trace, slurred speech, blood pressure, mean arterial pressure, or other suitable vitals. In some embodiments, calculating vitals based on the audio file or audio signal further includes requesting utterance of one or more vowel sounds at a specific frequency range and/or energy level with or without an example template, and/or computing robustness of the vowel utterance(s) by comparing them to an example template.

At Operation 307 the calculated vitals are provided to a medical practitioner. The method 300 ends at Operation 308 where the API removes itself from the call.

In some embodiments, the API 215 is further configured to identify slurring, patterns or abnormalities in the received audio file or audio signal. For further information and details on identifying slurred speech see Mani Sekhar et al., “Dysarthric-speech detection using transfer learning with convolutional neural networks”, ICT Express, Volume 8, Issue 1, 2022, Pages 61-64, and Canter et al., “Speech Characteristics of Patients with Parkinson's Disease: III. Articulation, Diadochokinesis, and Over-All Speech Adequacy”, Journal of Speech and Hearing Disorders, Volume 30, Number 3, Pages 217-224, 1965, each incorporated herein by reference in their entirety.

In some embodiments, the API 215 is further configured to calculate a score indicative of trauma, infection, or cardiac distress. In some embodiments, the API 215 is further configured to provide the score to a medical practitioner.

In some embodiments, the API 215 automatically intercedes the call. In some embodiments, the API 215 intercedes the call after an operator directs the API to intercede.

In some embodiments, the method 300 can further include providing a test tone or an appropriate synthetic human vocal sound such as, but not limited to, a vowel sound through the user's phone to assist the user in articulating a quasi-normalized vowel sound in both “pitch” (fundamental frequency) and “loudness” (amplitude) as a form of signal conditioning prior signal analysis. In some embodiments, the method 300 further utilizes a fundamental frequency detector and/or amplitude envelope detector to determine if the vocal utterances have been properly articulated including user feedback to “try again,” “louder,” “softer,” etc. In some embodiments, the signal is then subject to low frequency analysis via time-domain and/or frequency domain analysis, filtering, and/or low frequency oscillation detection for automatic, remote heartbeat pulse detection.

In some embodiments, the method 300 can further include using one or more on-board microphones of the user's device, such as a smartphone and placing the device near the heart thereby exploiting superior acoustic sound propagation solids and fluids when compared to propagation in the air. In some embodiments, external environmental noise is blocked while internal heartbeat/pulse sounds are maximally captured by the microphone. In some embodiments, the signal is then subject to low frequency analysis via time-domain and/or frequency domain analysis, filtering, and/or low frequency oscillation detection for automatic, remote heartbeat pulse detection.

### Method for Remote Patient Monitoring

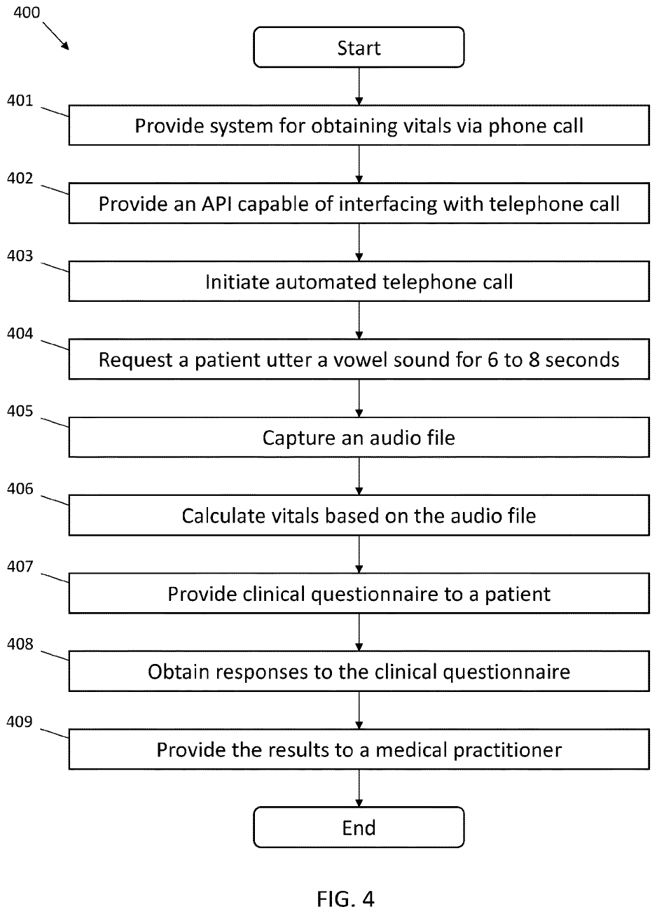

Referring now to FIG. 4, an exemplary method 400 for remote patient monitoring is shown. In some embodiments, the method 400 is configured to perform remote patient monitoring (RPM) and/or emergency triage. The method 400 starts at Operation 401 where a system for obtaining vitals via phone call such as system 200 is provided. At Operation 402, an API 215 configured to interface with an automated telephone call is provided.

In some embodiments, the interfacing is performed at the switch/exchange level using an existing public switched telephone network (PSTN) infrastructure for handoffs such as, for example, call waiting and sequential calls. In some embodiments, the interfacing is performed at the private branch exchange (PBX) level local to an entity such as a hospital or medical facility. In some embodiments, the interfacing is performed via a voice over internet protocol (VoIP). In some embodiments, the interfacing is performed via an application on a mobile telephone, smart phone, or any other suitable smart portable device. In some embodiments, the interfacing is performed via an application on a desk phone, computer, or similar device. In some embodiments, the interfacing is performed at a cloud based switch/exchange level such as Twilio, for example.

At Operation 403 an automated telephone call is initiated by the API 215. At Operation 404 a request is sent to a patient to utter a vowel sound for a set duration such as, for example, a duration in the range of 1 second to 20 seconds, 5 seconds to 10 seconds, 6 seconds to 8 seconds, about 7 seconds, or any other suitable duration. At Operation 405, an audio file or audio signal is captured. The audio file can be any suitable audio file (.wav, .mp3, or similar) or audio signal which includes the uttered vowel speech of a set duration.

At Operation 406 vitals are calculated based on the audio file or audio signal. In some embodiments, vitals are calculated as described above. In some embodiments the calculated vitals include one or more of heart rate, lung capacity, oxygen saturation, ECG trace, slurred speech, blood pressure, mean arterial pressure, or other suitable vitals. In some embodiments, calculating vitals based on the audio file or audio signal further includes requesting utterance of vowel at specific frequency range and/or energy level with or without an example template, and/or computing robustness of the vowel utterance by comparing it to the example template.

At Operation 407 a clinical questionnaire is provided to the patient. In some embodiments, the questionnaire is provided via text or audio. At Operation 408 responses to the questionnaire are obtained. The method 400 ends at Operation 409 where the calculated vitals and questionnaire responses are provided to a medical practitioner. In some embodiments, the API 215 is further configured to initiate clinical follow-up notes. In some embodiments, the questionnaire can be used in combination with the vitals to provide indication of progress or decline of a patients'condition.

In some embodiments, the API 215 is further configured to identify slurring, patterns or abnormalities in the received audio file or audio signal. For further information and details on identifying slurred speech see Mani Sekhar et al., “Dysarthric-speech detection using transfer learning with convolutional neural networks”, ICT Express, Volume 8, Issue 1, 2022, Pages 61-64, and Canter et al., “Speech Characteristics of Patients with Parkinson's Disease: III. Articulation, Diadochokinesis, and Over-All Speech Adequacy”, Journal of Speech and Hearing Disorders, Volume 30, Number 3, Pages 217-224, 1965, each incorporated herein by reference in their entirety.

In some embodiments, the API 215 is further configured to calculate a score indicative of trauma, infection, or cardiac distress. In some embodiments, the API 215 is further configured to provide the score to a medical practitioner.

In some embodiments, the method 400 can further include providing a test tone, human voice recording, or an appropriate synthetic human vocal sound such as, but not limited to, a vowel sound, through the user's phone to assist the user in articulating a quasi-normalized vowel sound in both “pitch” (fundamental frequency) and “loudness” (amplitude) as a form of signal conditioning prior signal analysis. In some embodiments, the method 300 further utilizes a fundamental frequency detector and/or amplitude envelope detector to determine if the vocal utterances have been properly articulated including user feedback to “try again,” “louder,” “softer,” etc. In some embodiments, the signal is then subject to low frequency analysis via time-domain and/or frequency domain analysis, filtering, and/or low frequency oscillation detection for automatic, remote pulse detection.

In some embodiments, the method 400 can further include using the on-board microphone of the user's device, such as a smartphone and placing the device near the heart thereby exploiting superior acoustic sound propagation solids and fluids when compared to propagation in the air. In some embodiments, external environmental noise is blocked while internal heartbeat/pulse sounds are maximally captured by the microphone. In some embodiments, the signal is then subject to low frequency analysis via time-domain and/or frequency domain analysis, filtering, and/or low frequency oscillation detection for automatic, remote heartbeat pulse detection.

### Non-Transitory Computer Readable Medium

In some embodiments, a non-transient computer readable medium is provided, storing instructions that, when executed by a computing system, cause the computer system connected to a telephonic communication system to host an application programming interface (API) configured to intercede into or interface with a call on the telephonic communication system to perform steps via the computing system comprising, requesting a patient to utter a sound for a set duration, capturing an audio file or audio signal, and/or calculating vitals based on the audio file or audio signal.

In some embodiments, the interceding is performed at the switch/exchange level using an existing public switched telephone network (PSTN) infrastructure for handoffs such as, for example, call waiting and sequential calls. In some embodiments, the interceding is performed at the private branch exchange (PBX) level local to an entity such as a hospital or medical facility. In some embodiments, the interceding is performed via a voice over internet protocol (VoIP). In some embodiments, the interceding is performed via an application on a mobile telephone, smart phone, or any other suitable smart portable device. In some embodiments, the interceding is performed via an application on a desk phone, computer, or similar device. In some embodiments, the interceding is performed at a cloud based switch/exchange level such as Twilio, for example.

In some embodiments, the calculated vitals comprise at least one of heart rate, lung capacity, oxygen saturation, ECG trace, slurred speech, blood pressure, and mean arterial pressure.

### Algorithm Description

Exemplary details of algorithms used in the above methods are described below.

While specific details are described, additional algorithmic steps not described can also be utilized, and those steps that are described may be optional, modified, or performed in an order different from that described as one skilled in the art would understand.

In some embodiments, an individual is prompted to hold a vowel sound, e.g.“aahhh,” for 6 to 8 seconds. Once the audio sample is recorded, the data is read into a one-dimensional array. A 16^{th} order Finite Impulse Response (FIR) Band Pass filter is applied to the signal in an effort to reduce computation on undesired frequencies. The pass band of the filter may have a lower bound of between 0.01 Hz and 5 Hz, or between 0.01 Hz and 1 Hz, or between 0.01 Hz and 0.5 Hz, or between 0.01 Hz and 0.3 Hz, or between 0.01 Hz and 0.1 Hz, or between 0.01 Hz and 0.05 Hz, or about 0.04 Hz or about 0.03 Hz. The pass band of the filter may have an upper bound of between 100 Hz and 300 Hz, or between 120 Hz and 280 Hz, or between 140 Hz and 260 Hz, or between 160 Hz and 240 Hz, or between 180 Hz and 220 Hz, or between 190 Hz and 210 Hz, or about 200 Hz. In some embodiments, a low-pass filter may be used, having an upper bound as described.

In some embodiments, a Short-Time Fourier Transform (STFT) is then applied to the filtered signal. This process segments the signal into windows of 2048 samples with an overlap of 1800 samples, forming a two-dimensional (2-D) matrix of pixels, each having an intensity value. Each row in this matrix is transformed by a Fast-Fourier Transform (FFT) in order to reveal changes in frequency components of the audio samples over time.

In some embodiments, to reduce background noise in the STFT, a one-sided threshold filter is applied to suppress pixels with intensity less than 10% of the maximum brightness. This can effectively reduce side talk noise from the environment.

In some embodiments, in an effort to narrow down the search for heart rate related frequencies, an additional FIR Band Pass filter is applied. The FIR Band pass filter may be a 4^{th} order, 5^{th} order, 6^{th} order, 7^{th} order, 8^{th} order, 9^{th} order, 10^{th} order, 11^{th} order, 12^{th} order, 13th order, 14^{th} order, 15^{th} order, 16^{th} order, 17^{th} order, 18^{th} order, 19^{th} order, or 20^{th} order FIR Band pass filter. The pass band of this filter may for example be between 0.67 Hz and 3.33 Hz, corresponding to the extremes of the human heart beat, 40 beats per minute (bpm) to 200 bpm. This filter may be applied to some or all bins of the STFT.

In some embodiments, each bin of the STFT is then passed through another FFT in order to reveal periodicity in the frequency information of the audio sample.

In some embodiments, the rows of the spectrum are then summed vertically in an effort to amplify periodic harmonics that are present.

In some embodiments, the search range of harmonics is between 0.67 Hz and 3.33 Hz, which is the range of the human heart beat, 40 bpm to 200 bpm.

In some embodiments, a peak detection algorithm is then implemented in this range of frequencies in order to find harmonic peaks in the spectrum.

In some embodiments, to understand which peaks belong to the heart rate of the individual, constraints are implemented to identify exactly which peaks are related.

In some embodiments, the distance between each peak to each other peak is calculated without repetition. If a distance falls outside the range 40 bpm to 200 bpm, it is not related to the heart rate.

Furthermore, in some embodiments, if the distance is not equal to one of the peaks detected, it is not related to the heart rate.

In some embodiments, the value that is most common (i.e. the mode) within the distances and peaks detected is taken to be the heart rate of the individual. As these values are not exact and could vary by ±5 bpm, an average of the most common distances and peaks detected may be used as the heart rate of the individual. In some embodiments, the values may be binned in ±5 bpm, ±3 bpm, ±2 bpm, or ±1 bpm bins, and the bin having the most elements may be used as the heart rate of the individual.

The aforementioned systems, processes and methods described herein may be utilized for desired practical applications as would be appreciated by those skilled in the art.

For example, the systems and methods presented herein can be used to perform asynchronous cardiac monitoring or remote triage for emergency and non-emergency medical events.

The following publications are each hereby incorporated herein by reference in their entirety:
Mesleh A., Skopin D., Baglikov S., and Quteishat A., Heart rate extraction from vowel speech signals. JOURNAL OF COMPUTER SCIENCE AND TECHNOLOGY 27(6): 1243-1251 Nov. 2012. DOI 10.1007/s11390-012-1300-6
S. R. Mani Sekhar, Gaurav Kashyap, Akshay Bhansali, Andrew Abishek A., and Kushan Singh, Dysarthric-speech detection using transfer learning with convolutional neural networks, ICT Express, Volume 8, Issue 1, 2022, Pages 61-64, ISSN 2405-9595, https://doi.org/10.1016/j.icte.2021.07.004.
Gerald J. Canter, Speech Characteristics of Patients with Parkinson's Disease: III. Articulation, Diadochokinesis, and Over-All Speech Adequacy, Journal of Speech and Hearing Disorders, Volume 30, Number 3, Pages 217-224, 1965, Doi:10.1044/jshd.3003.217, https://pubs.asha.org/doi/abs/10.1044/jshd.3003.21

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A system for calculating vitals via phone call, comprising:
a computing system communicatively connected to a telephonic communication system, comprising a processor and a non-transitory computer-readable medium with instructions stored thereon, which when executed by the processor, host an application programming interface (API) configured to intercede into or interface with a call on the telephonic communication system to perform steps via the computing system comprising:
requesting a patient utter a sound for a set duration;
capturing an audio file or audio signal; and
calculating vitals based on the audio file or audio signal.

2. The system of claim 1, wherein the step of calculating vitals based on the audio file or audio signal comprises:
trimming the audio file or audio signal to a set timeframe or duration;
performing digital signal processing including time-domain, frequency-domain, and/or spectral analysis to obtain a spectrogram;
analyzing the waveform and the spectrogram for patterns in a defined frequency or magnitude range;
graphing an electrocardiogram (ECG) based on the analysis;
passing the ECG through a filtering process to produce a filtered ECG;
detecting peaks or salient resonance points in the filtered ECG to obtain frequency values; and
calculating a heart rate based on the frequency values obtained.

3. The system of claim 1, wherein the calculated vitals comprise at least one of heart rate, lung capacity, oxygen saturation, ECG trace, slurred speech, blood pressure, and mean arterial pressure.

4. The system of claim 1, wherein the API further performs steps via the computing system comprising:
providing the calculated vitals to a medical practitioner; and
removing itself from the call.

5. The system of claim 1, wherein the API further performs steps via the computing system comprising:
initiating an automated telephone call;
providing a clinical questionnaire via the automated telephone call or a text message;
obtaining responses to the clinical questionnaire via the automated telephone call or the text message; and
providing the calculated vitals and the responses to the clinical questionnaire to a medical practitioner.

6. The system of claim 1, further comprising a database communicatively connected to the computing system.

7. The system of claim 6, wherein the API via the computing system is further configured to store the audio file or audio signal feature vectors, algorithmic parameters, or calculated vitals on the database.

8. A method for obtaining vitals via phone call, comprising:
providing the system of claim 1; and
interceding into or interfacing with a phone call via an application programming interface (API) of the computing system to perform steps via the computing system comprising:
sending a request to a patient to utter a sound for a set duration;
capturing an audio file or audio signal; and
calculating vitals based on the audio file or audio signal.

9. The method of claim 8, wherein the step of calculating vitals based on the audio file or audio signal comprises:
trimming the audio file or audio signal to a set timeframe or duration;
performing digital signal processing including time-domain, frequency-domain, and/or spectral analysis to obtain a spectrogram;
analyzing the waveform and the spectrogram for patterns in a defined frequency or magnitude range;
graphing an electrocardiogram (ECG) based on the analysis;
passing the ECG through a filtering process to produce a filtered ECG;
detecting peaks or salient resonance points in the filtered ECG to obtain a frequency values; and
calculating a heart rate based on the frequency values obtained.

10. The method of claim 8, wherein the calculated vitals comprise at least one of heart rate, lung capacity, oxygen saturation, ECG trace, slurred speech, blood pressure, and mean arterial pressure.

11. The method of claim 8, wherein the API further performs steps via the computing system comprising:
providing the calculated vitals to a medical practitioner; and
removing itself from the call.

12. The method of claim 8, wherein the API further performs steps via the computing system comprising:
initiating an automated telephone call;
providing a clinical questionnaire via the automated telephone call or a text message;
obtaining responses to the clinical questionnaire via the automated telephone call or the text message; and
providing the calculated vitals and the responses to the clinical questionnaire to a medical practitioner.

13. The method of claim 8, wherein the API via the computing system is further configured to identify slurring, patterns or abnormalities in the audio file or audio signal.

14. The method of claim 8, wherein the API via the computing system is further configured to calculate a score indicative of trauma, infection, or cardiac distress.

15. The method of claim 14, wherein the API via the computing system is further configured to provide the score to a medical practitioner.

16. The method of claim 8, wherein the API via the computing system automatically intercedes the call.

17. The method of claim 8, wherein the API via the computing system intercedes the call after an operator initiates the API to intercede.

18. The method of claim 8, wherein the API via the computing system is further configured to initiate clinical follow-up notes.

19. A non-transitory computer readable medium storing instructions that, when executed by a computing system, cause the computer system connected to a telephonic communication system to host an application programming interface (API) configured to intercede into or interface with a call on the telephonic communication system to perform steps via the computing system comprising:
requesting a patient utter a sound for a set duration;
capturing an audio file or audio signal; and
calculating vitals based on the audio file or audio signal.

20. The non-transitory computer readable medium of claim 19, wherein the calculated vitals comprise at least one of heart rate, lung capacity, oxygen saturation, ECG trace, slurred speech, blood pressure, and mean arterial pressure.
